(19) United States
(12) Patent Application Publication
(10) Pub. No.: US 2026/0248604 A1
(43) Pub. Date: Aug. 27, 2026

(54) PLICATION SYSTEMS, PLICATION/SPRING SYSTEMS, AND METHODS OF USE THEREOF

(71) Applicant: ECLIPSE REGENESIS, INC., Mountain View, CA (US)

(72) Inventors: Andrew Lantz, Redwood City, CA (US); Bernie Andreas, Los Altos, CA (US); Andre Bessette, San Mateo, CA (US); James Dunn, Palo Alto, CA (US); James Wall, Woodside, CA (US); Roberto Gugig, Palo Alto, CA (US)

(21) Appl. No.: 18/857,788

(22) PCT Filed: Apr. 18, 2023
(86) PCT No.: PCT/US2023/018944

§ 371 Date: Oct. 17, 2024

Related U.S. Application Data
(60) Provisional application No. 63/332,230, filed on Apr. 18, 2022.

(51) Int. Cl. A61F 2/04

(52) U.S. Cl. CPC A61F 2/04; A61F 2002/045; A61F 2250/0065

(57) ABSTRACT

Plicator systems may have two telescoping components that when arranged in an open configuration have one or more windows into which tissue of, for example, a tubular organ may be invaginated. The invaginated tissue may be secured in the window when the plicator system transitions to a closed configuration. Closing the window may act to, for example, clamp, pierce of the tubular organ tissue invaginated into the window, thereby securing the invaginated tubular organ tissue into the window. In some instances, an axially-expanding spring may be disposed between two plicator systems and when this assembly is implanted in a tubular organ, axial expansion of the spring may exert a distending force on the tubular organ, which may stimulate growth of the tubular organ.

### RELATED APPLICATION

This application is a patent application filed under the Patent Cooperation Treaty and claims priority to U.S. Provisional Patent Application No. 63/332,230, which was filed on 18 Apr. 2022 and entitled “PLICATION SYSTEMS, PLICATION/SPRING SYSTEMS, AND METHODS OF USE THEREOF” and is incorporated, in its entirety, herein.

### TECHNICAL FIELD

The disclosure herein generally relates to plication systems for use with tubular organs and, more particularly to plication/spring systems and delivery devices and methods of using same to facilitate growth of and/or elongate tubular organs.

### BACKGROUND

Short bowel syndrome (SBS) is a syndrome caused by a shortened, or dysfunctional, small intestine that impairs the body's ability to adequately digest food and/or absorb enough nutrients from ingested food to sustain the body. Medical treatment for SBS includes administration of parenteral nutrition to provide necessary nutrients and hydration. Surgical treatment options for SBS include intestinal transplantation, procedures that taper and lengthen the intestine to increase absorption area and procedures that slow down transit time for food and/or nutrients traveling through the small intestine. Recently, the concept of using mechanical force to lengthen intestinal tissue has been studied using a variety of tissue expander devices. However, many of these methods require repeated invasive interventions such as serial screw advancement or saline injections. Techniques for distraction enterogenesis, where axial force is applied by springs implanted in the small bowel, have also been employed to create increased intestinal length.

### SUMMARY

Plicator systems configured for implantation within a tubular organ as disclosed herein may include telescoping cylindrical inner and outer components that may be configured to fit together and articulate relative to one another from and open to a closed position. The outer component may include a first lumen, a first notch, and a retention mechanism. In some embodiments, the outer component may include one or more teeth that extend into the first notch. The teeth may be configured to engage with and hold the invaginated tubular organ tissue within the window via, for example, clamping and/or piercing of the tissue.

The retention mechanism may be one or more of, for example, a clip mechanism configured to engage with one or more projections of the plurality of projections as, for example, the plicator system translates from the open configuration to the closed configuration that, in some cases, may lock the inner component in place relative to outer component. Additionally, or alternatively, the retention mechanism may be a ratcheting mechanism and/or include a cantilevered snap configured to engage with a projection of the plurality of projections and maintain a position of inner component relative to outer component.

The inner component may include a second lumen, a second notch, and a plurality of projections extending from an outer surface of the inner component, each of the plurality of extensions being arranged and configured to engage with the retention mechanism. A portion of the inner component may be disposed within the outer component so that the first lumen aligns with the second lumen, thereby generating a central lumen for the plicator system. At times, the inner component may include one or more teeth that extend into the second notch, the one or more teeth being configured to engage with and hold the invaginated tubular organ tissue within the window via, for example, clamping and/or piercing of the tissue. A size of the one or more teeth of the inner component and/or the outer component may be responsive to at least one of a size and a thickness of the tubular organ.

The first notch and the second notch may be arranged and configured to form a window with an open area into which a portion of tubular organ tissue may be invaginated when the plicator system is implanted into the tubular organ and the plicator system may be arranged in an open configuration The plicator system may be configured to translate from the open configuration to a closed configuration as the inner component may be pushed toward outer component, thereby reducing a size of the open area of the window and clamping, and/or trapping, the invaginated tubular organ tissue therein so that the plicator system may be securely implanted within the tubular organ.

In some embodiments, the plicator system may include an occluding component that may be attached to an end of the inner component not disposed within the outer component and configured to occlude the second lumen. Additionally, or alternatively, the plicator system may include a delivery extension that may be attached to an end of the inner component not disposed within the outer component. The delivery extension may be configured for cooperation with a plicator system delivery device.

Plicator and spring system disclosed herein may include a first plicator system, a second plicator system, a spring disposed between, and attached to, the first and second plicator systems. The first and second plicator systems may have the same, similar, and/or different dimensions compared with one another. The spring may have a tubular body with a lumen therethrough that aligns with the lumens of the first and second plicator systems. The spring may be configured to axially expand, thereby pushing the first plicator system away from the second plicator system.

In some instances, the plicator and spring system may also include an occluding component that is attached to an end of the first inner component not disposed within the first outer component and configured to occlude the second lumen. Additionally, or alternatively, the plicator and spring system may include a delivery extension that is removably attached to an end of the second inner component not disposed within the second outer component and configured to attach to a delivery device for the plicator and spring system. In these embodiments, the plicator and spring system may include a delivery device configured to coupled to the delivery extension. The delivery device may be configured to facilitate invagination of tubular organ tissue into the first window of the first plicator system and the second window of the second plicator system and facilitate closing of the first window and the second window, thereby securing the tubular organ tissue invaginated into the first window and the second window therein.

Methods for implanting a plicator and spring system into a tubular organ of a subject may include positioning the plicator and spring system within the tubular organ, applying negative pressure (e.g., vacuum or suction) to the plicator and spring system, thereby pulling tubular organ tissue into a window of the plicator and spring system, and actuating the plicator and spring system to secure the tubular organ tissue within the window via, for example, clamping and/or piercing of the tissue. The method may be performed by, for example, a health care provider such as a surgeon or doctor. When the plicator and spring system includes an occluding component configured to occlude the lumen of the first and/or second plicator and spring systems, the method may further include removing the occluding component following implantation of the plicator and spring system within the tubular organ. Delivery of the plicator and spring system to a target site in the subject's tubular organ may be performed using, for example, a delivery device and/or an inserter configured to couple to a delivery extension of the plicator and spring system.

### BRIEF DESCRIPTION OF THE DRAWINGS

The accompanying drawings, which are incorporated in and constitute a part of this disclosure, illustrate exemplary embodiments and, together with the description, serve to explain the disclosed invention.

FIG. 1A1 is a front side view of a plicator system, the back side view being a mirror image thereof, in accordance with some embodiments of the present invention.

FIG. 1A2 is a top view of the plicator system as oriented in FIG. 1A1, in accordance with some embodiments of the present invention.

FIG. 1A3 is a bottom view of the plicator system as oriented in FIG. 1A1, in accordance with some embodiments of the present invention.

FIG. 1B1 is a right-side view of the plicator system, the left side view being a mirror image thereof, in accordance with some embodiments of the present invention.

FIG. 1B2 is a top view of the plicator system as oriented in FIG. 1B1, in accordance with some embodiments of the present invention.

FIG. 1B3 is a bottom view of the plicator system as oriented in FIG. 1B1, in accordance with some embodiments of the present invention.

FIG. 1C1 is a three-fourths side view of the plicator system, the one-fourth side view being a mirror image thereof, in accordance with some embodiments of the present invention.

FIG. 1C2 is a top view of the plicator system as oriented in FIG. 1C1, in accordance with some embodiments of the present invention.

FIG. 1C3 is a bottom view of the plicator system as oriented in FIG. 1C1, in accordance with some embodiments of the present invention.

FIG. 2A is a top view of the plicator system as shown in FIG. 1A2 with horizontal and vertical cross-section dividing lines superimposed thereon, in accordance with some embodiments of the present invention.

FIG. 2B is a horizontal cross-sectional view of the plicator system of FIG. 2A, in accordance with some embodiments of the present invention.

FIG. 2C is a vertical cross-sectional view of the plicator system of FIG. 2A, in accordance with some embodiments of the present invention.

FIG. 3A is a schematic representation of a spring that may be used with one or more of the plicator systems disclosed herein, in accordance with some embodiments of the present invention.

FIG. 3B is a schematic diagram of a spring when in a compressed state with three clips, in accordance with some embodiments of the present invention.

FIG. 3C is a schematic diagram of the spring of FIG. 3B with an expanded first segment, in accordance with some embodiments of the present invention.

FIG. 3D is a schematic diagram of the spring of FIG. 3B with an expanded first and second segment, in accordance with some embodiments of the present invention.

FIG. 3E is a schematic diagram of the spring of FIG. 3B when fully expanded, in accordance with some embodiments of the present invention.

FIG. 3F is a schematic representation of plicator and spring system with a first and a second plicator system positioned on either side of a spring, wherein the first and second plicator systems are in an open configuration and spring is in a compressed configuration, in accordance with some embodiments of the present invention.

FIG. 3G is a schematic representation of the plicator and spring system of FIG. 3F, wherein the first and second plicator systems and are in a closed configuration and spring is in a compressed configuration, in accordance with some embodiments of the present invention.

FIG. 3H is a schematic representation of the plicator and spring system of FIG. 3G with an occluding component removed therefrom, in accordance with some embodiments of the present invention.

FIG. 3I is a schematic representation of the plicator and spring system of FIG. 3H, wherein the spring is in an open, or expanded, configuration, in accordance with some embodiments of the present invention.

FIG. 4A is a photograph of tubular organ tissue invaginated into a window of a plicator system, in accordance with some embodiments of the present invention.

FIG. 4B is a photograph of tubular organ tissue invaginated into a window of a plicator and spring system that includes one plicator and one spring, in accordance with some embodiments of the present invention.

FIG. 5 is a schematic representation of a plicator and spring system coupled to a plicator and spring system delivery device, in accordance with some embodiments of the present invention.

FIG. 6 is a flowchart illustrating a process for treating a patient using a plicator and spring system, in accordance with some embodiments of the present invention.

FIG. 7A is a schematic diagram of an exemplary plicator and spring system configured for insertion into a tubular organ through an opening in the skin and/or via a stoma while in a compressed state, in accordance with some embodiments of the present invention.

FIG. 7B is a schematic diagram of a front view of a flange included in the exemplary plicator and spring system of FIG. 7A, in accordance with some embodiments of the present invention.

FIG. 7C is a schematic diagram of the exemplary plicator and spring system of FIG. 7A while in an expanded state, in accordance with some embodiments of the present invention.

FIG. 7D provides a side view of the plicator, spring, and flange system system of FIG. 7A when implanted in a subject's tubular organ and is in an unextended state, in accordance with some embodiments of the present invention.

FIG. 7E provides a side view of the plicator, spring, and flange system system of FIG. 7A when implanted in a subject's tubular organ and is in an extended state, in accordance with some embodiments of the present invention.

FIG. 7F is a schematic diagram of another exemplary plicator and spring system configured for insertion into a tubular organ through an opening in the skin and/or via a stoma while in a compressed state, in accordance with some embodiments of the present invention.

FIG. 7G provide is a schematic diagram of the exemplary plicator and spring system of FIG. 7F while in an expanded state, in accordance with some embodiments of the present invention.

FIG. 8 is a flowchart illustrating a process for treating a patient using a plicator and spring system configured insertion into a tubular organ through an opening in the skin and/or via a stoma, in accordance with some embodiments of the present invention.

FIG. 9 is a block diagram of a kit, in accordance with some embodiments of the present invention.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

Exemplary embodiments are described with reference to the accompanying drawings. In the figures, the leftmost digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. Reference throughout this specification to “one embodiment”, “this embodiment” and similar phrases, means that a particular feature, structure, or characteristic described in connection with the embodiment is included in at least one of the one or more embodiments. Thus, the appearances of these phrases in various places throughout this specification are not necessarily referring to the same embodiment. Furthermore, the particular features, structures, or characteristics may be combined in any suitable manner in one or more embodiments. Unless otherwise defined herein, scientific and technical terms used in connection with the present application shall have the meanings that are commonly understood by those of ordinary skill in the art to which this disclosure belongs.

Embodiments of present disclosure provide a plicator system(s), plicator and spring system(s), delivery device(s), kits including plicator and spring system(s) and delivery device(s), and method(s) for use thereof within tubular, or hollow, organs and/or tissue (collectively referred to herein as “tubular organ”) such as the small intestine, a urethra, and/or a fallopian tube for the purpose of stimulating growth (e.g., elongating) of the tubular organ via exertion of an expanding force on the tubular organ. The plicator and spring systems disclosed herein may be implanted within a tubular organ via, for example, laparoscopy, or by an open surgical procedure and secured along a length of the tubular organ by forming one or more plication(s) at each end of the plicator and spring system to secure the plicator and spring system to the tubular organ tissue via, for example, clamping and/or piercing of the tissue as described herein.

A number (e.g., 1-5) of plicator and spring system(s) placed in the tubular organ of a patient may depend on a variety of factors such as the tubular organ of implantation, the desired amount of tubular organ lengthening, and/or the type of plicator and spring system being employed. When multiple plicator and spring systems are used in a patient, they may be inserted in the tubular organ of the patient concurrently in, for example, a series along a length of a portion of the tubular organ and/or serially over time. When multiple plicator and spring systems are implanted within a tubular organ concurrently, they may be distributed evenly and/or unevenly along the length of the tubular organ.

The plication systems and plicator and spring systems disclosed herein generally include a proximal end, a distal end, an expanded configuration, and a compressed configuration. Although typically axially expandable, the plication systems and/or plicator and spring systems may also expand in the radial direction to, for example, expand a diameter of the tubular organ. In some variations, the plicator and spring systems disclosed herein may expand over a period of time that may range from, for example, 1-8 weeks to apply a steady expansion force to the tubular organ into which it is implanted and thereby stimulate growth of the proximately situated tubular organ.

In one embodiment, the plicator and spring systems disclosed herein may be placed using a delivery device via an open surgical procedure by cutting the tubular organ and surrounding tissue, inserting the plicator and spring system(s) into the cut tubular organ, applying a vacuum to the plicator and spring system(s) to pull tissue into openings, or windows positioned within the plicator and spring systems so that the tissue may be grabbed by the teeth positioned within the windows, and finally closing plicator systems of the plicator and spring system to secure the tissue within the window(s) via, for example, clamping and/or piercing of the tissue. The plicator and spring system may then be released from the delivery device, the delivery device may be removed from the patient's body and the surgical opening may be closed. In another embodiment, the device may be placed via a minimally invasive manner via, for example, an endoscopic procedure. In another embodiment, where a portion of the tubular organ extends to, or outside of, the patient's epidermis thereby creating a stoma (as may be the case with patients whose small intestine extends beyond the abdominal wall to, for example, facilitate feeding through a feeding tube), the plicator and spring system(s) disclosed herein may be placed within the tubular organ through the stoma.

The plicator and spring systems disclosed herein may be surgically removed when, for example, they have fully expanded and/or the tubular organ has been expanded to a desired length. Alternatively, the plicator and spring systems disclosed herein be sloughed off via natural tissue growth of the tubular organ and pass through the patient's digestive tract.

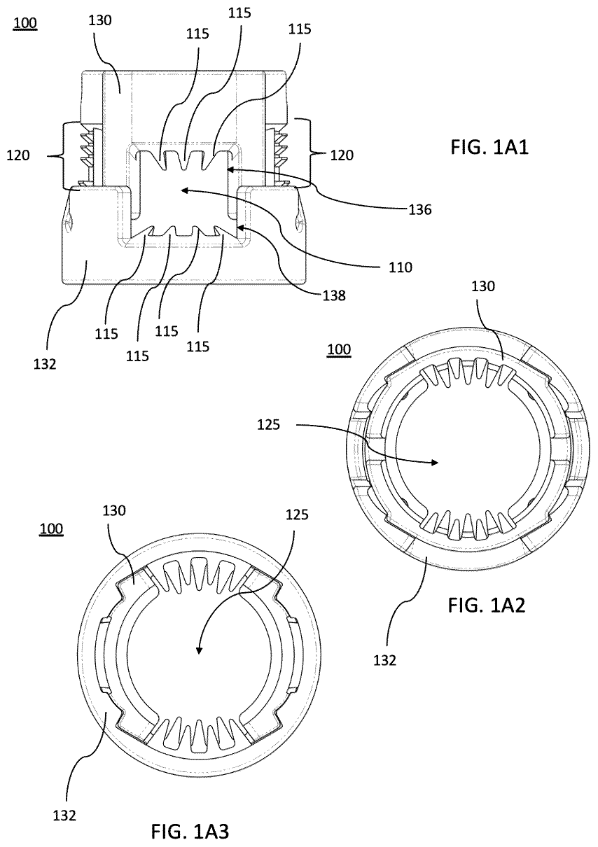

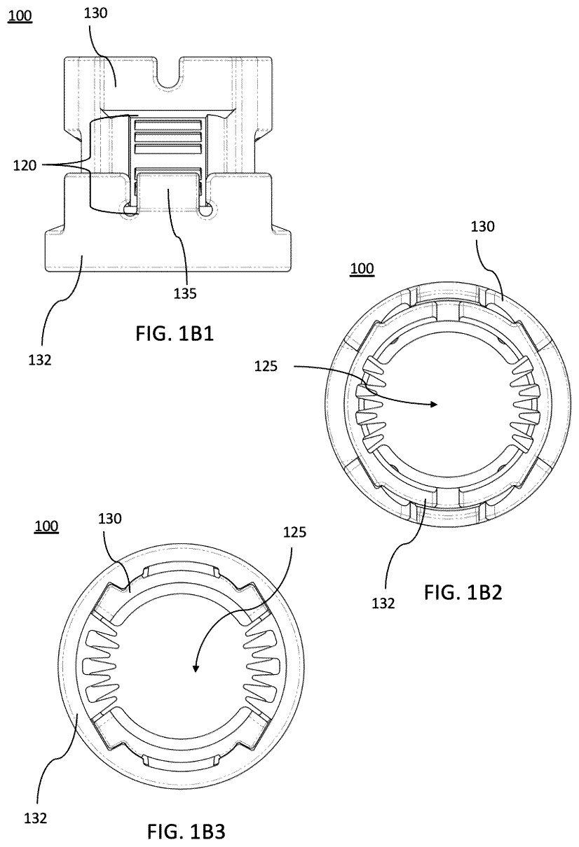

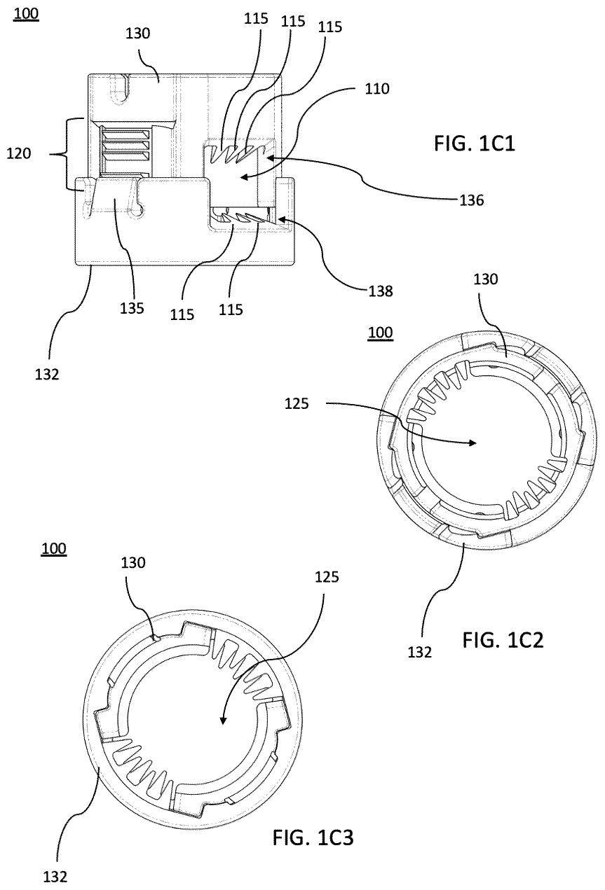

Referring now to the drawings, FIG. 1A1-1C3 provide schematic diagrams of various views of a plicator system 100 that includes an inner component 130 and an outer component 132 with varying diameters that cooperate as telescoping cylinders that, in some cases, may allow for small compressed/actuation width and a closed vacuum volume. Additionally, or alternatively, plicator system 100 may be and/or include interlocking hollow discs.

In particular, FIG. 1A1 is a front side view of plicator system 100, wherein the back side view is a mirror image thereof, FIG. 1A2 is a top view of plicator system 100 as oriented in FIG. 1A1, FIG. 1A3 is a bottom view of plicator system 100 as oriented in FIG. 1A1, FIG. 1B1 is a right side view of the plicator system 100, wherein the left side view is a mirror image thereof, FIG. 1B2 is a top view of the plicator system 100 as oriented in FIG. 1B1, FIG. 1B3 is a bottom view of the plicator system 100 as oriented in FIG. 1B1, FIG. 1C1 is a three-fourths side view of the plicator system 100, wherein the one-fourth side view is a mirror image thereof, FIG. 1C2 is a top view of the plicator system 100 as oriented in FIG. 1C1, and FIG. 1C3 is a bottom view of the plicator system 100 as oriented in FIG. 1C1.

As may be seen in FIG. 1A1, inner component 130 includes a first notch 136 with three projections, or teeth, 115 positioned along an upper edge (as oriented in FIG. 1A1) projecting into the empty space formed by first notch 136. An exterior of inner component has an array of projections 120 configured to cooperate with a locking and/or retention mechanism 135 (as will be discussed in greater below with regard to FIG. 1B1) to keep inner component 130 at a desired position relative to outer component 132. In one embodiment, array of projections 120 and/or locking and/or retention mechanism 135 may be embodied to include a ratcheting locking mechanism. Other variations for array of projections 120 and/or locking and/or retention mechanism 135 may include a threaded/screw mechanism, a friction and/or press fit mechanism, an annular snap, a j-lock mechanism and/or a latching mechanism.

Outer component 132 has a second notch 138 with four teeth 115 positioned along a lower edge (as oriented in FIG. 1A1) projecting into the empty space formed by second notch 138. When inner component 130 is seated within outer component 132 as shown in FIG. 1A1, a window 110 is positioned therebetween. Window 110 may be configured to accept invagination of a portion of a tubular organ tissue therein and teeth 115 may be configured and arranged to hold the invaginated tissue within window 110 via, for example, clamping and/or piercing of the tissue as, for example, shown and described herein.

As may be seen in FIG. 1A2,1A3, 1B2, 1B3, 1C2, and 1C3, plicator system 100 may have a lumen 125 positioned in an approximate center thereof. Lumen 125 may be configured to allow material (e.g., water, partially digested food, etc.) to pass therethrough so that, for example, functioning of a tubular organ is not impaired by implantation of plicator system 100 or a device including plicator system 100.

As may be seen in FIG. 1B1, plicator system 100 includes locking and/or retention mechanism 135 that is configured to engage via a clip mechanism with one or more of the projections of projection array 120 and maintain a position of inner component 130 with regard to outer component 132. FIG. 1C1 shows a relative position between window 110, teeth 115, projection array 120, and locking and/or retention mechanism 135, as they are arranged circumferentially around inner component 130 and outer component 132, respectively.

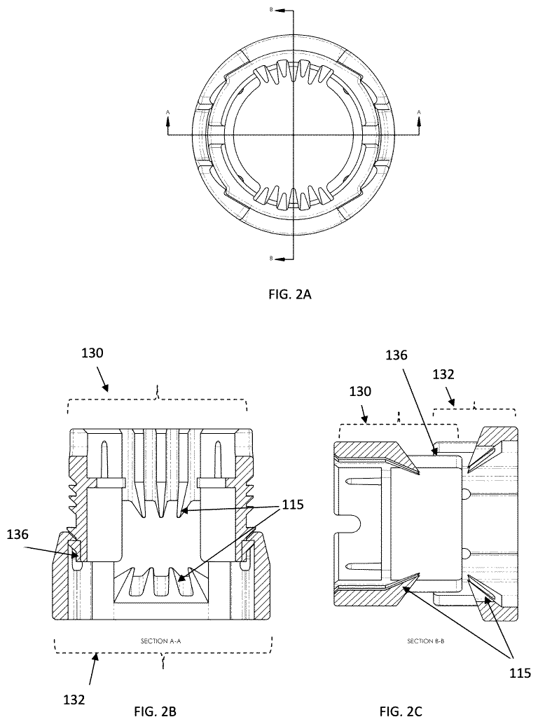

FIG. 2A is a top view of the plicator system as shown in FIG. 1A2 with a horizontal cross-section dividing line A-A and a vertical cross-section dividing line B-B superimposed thereon, FIG. 2B is a horizontal cross sectional view taken at line A-A and going from a top to the bottom (as oriented in FIG. 1A1), of plicator system 100 and FIG. 2C is a vertical cross sectional view of the plicator system taken at line B-B and going from a top to the bottom (as oriented in FIG. 1A1), of plicator system 100. As may be seen in FIG. 2B, inner component 130 and outer component 132 are interlocked at position 142 with two locking and/or retention mechanisms 135 positioned on either side (i.e., 180 degrees from each other) of outer component 132, that, in this embodiment are configured as two cantilevered snaps 150 to engage with an projection of projection array 120 to hold outer component 132 in place relative to inner component 130 as shown.

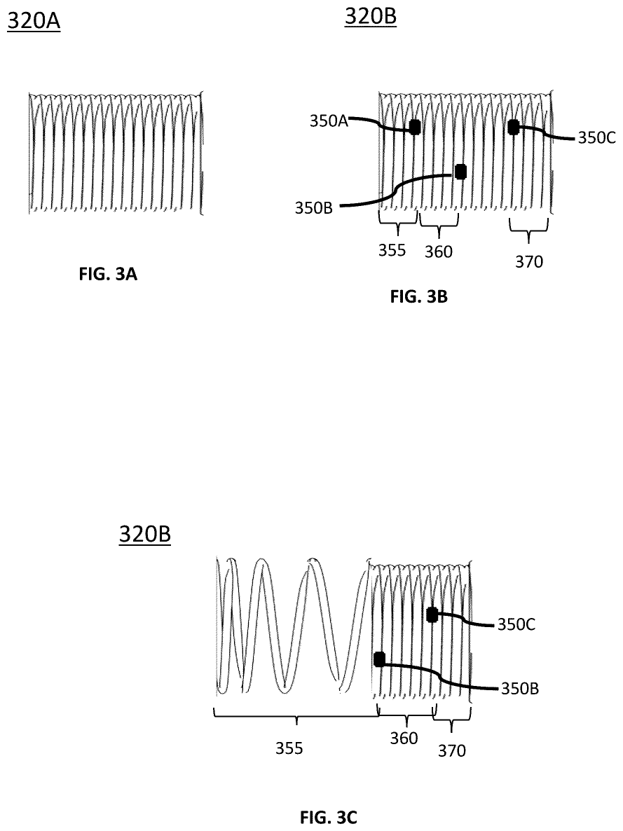

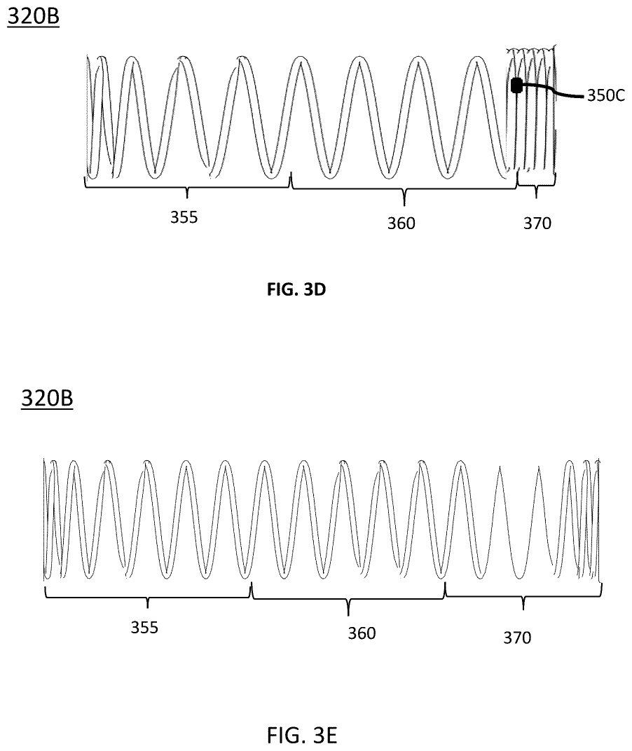

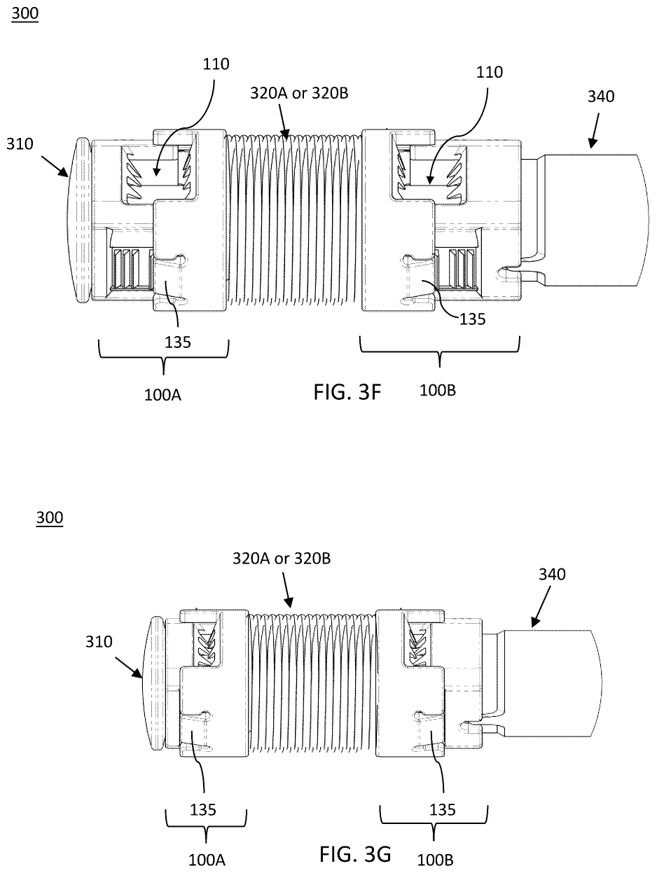

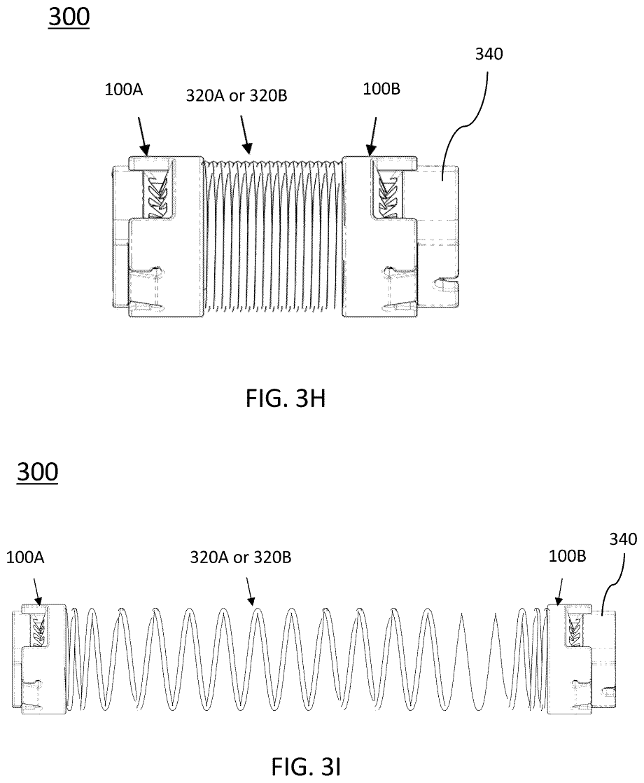

FIG. 3A is a schematic representation of a spring 320A that may be used with one or more of the plicator systems disclosed herein. In many embodiments, spring 320A may be an axially expanding hollow tubular body with a lumen therethrough that aligns with lumen 125 and maintains the lumen of a tubular organ in which it is implanted so that bodily contents (e.g., digested food and/or bodily fluids) may pass therethrough. Spring 320A may be structured as, for example, a hollow tube, expanding polymer, coil, and/or spring. Additionally, or alternatively, spring 320A may comprise/include a braided or woven stent-like structure that may be made from, for example, filaments or wires.

In some embodiments, spring 320A may include a single release point that extends like a spring or may include multiple release points that, for example, use bioabsorbable clips and/or retention mechanisms 350 to keep a spring like spring 320B in a compressed state as shown in FIG. 3B and then released resulting in a multi-stage component extension as shown in, for example, FIGS. 3C-3E. In particular, FIG. 3B is a schematic diagram of a spring 320B when in a compressed state. Spring 320B is similar to spring 320A with the exception that it includes a first clip 350A, a second clip 350B, and a third clip 350C positioned along the length of spring 320B as shown. First, second, and third clips 350A, 350B, and 350C divide spring 320B into a first segment 355, a second segment 360, and a third segment 370. First, second, and/or third clips 350A, 350B, and/or 350C may be made from a bioabsorbable and/or biodegradeable compound and may function so that when they are absorbed and/or degraded after implantation in the body, a force preventing axial expansion of spring 320B may be released and spring 320B may expand. In some instances, first, second, and third clips 350A, 350B, and 350C may be configured to absorb and/or degrade at different rates so that the axial expansion of spring 320B is performed in a gradual manner. In one example, first clip 350A may be configured to degrade and/or be absorbed first (e.g., 1-14 days following inplantation), which may release first segment 355 of spring 320B as shown in FIG. 3C; second clip 350B may be configured to degrade and/or be absorbed following absorption of first clip 350A (e.g., 3-20 days following inplantation), which may release second segment 360 of spring 320B as shown in FIG. 3D; and third clip 350C may be configured to degrade and/or be absorbed last (e.g., within 6-30 days following inplantation), which may release third segment 370 of spring 320B as shown in FIG. 3E. Following absorption of third clip 350C, spring 320B may also release a third segment 370 so that spring 320B is fully expand as shown in FIG. 3E.

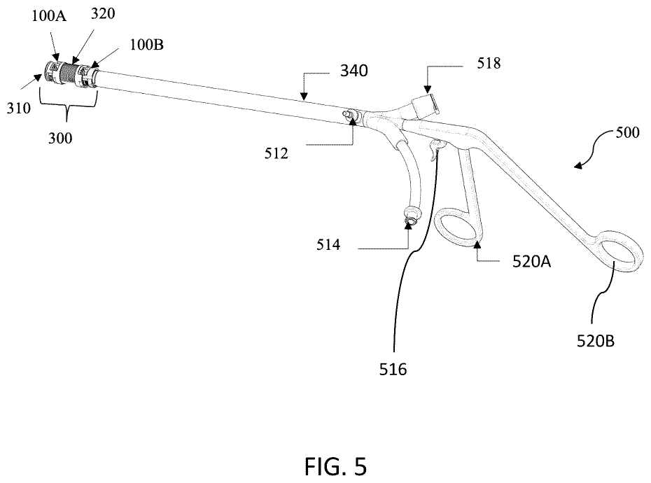

FIG. 3F is a schematic representation of a plicator and spring system 300 that includes a first plicator system 110A and a second plicator system 100B positioned on either side of spring 320A or 320B, wherein the first and second plicator systems 100A and 100B are in an open configuration and spring 320A or 320B is in a compressed configuration. Plicator and spring system 300 also includes an occluding component 310 positioned at an end of first plicator system 100A. FIG. 3F also shows a portion of a delivery extension, or inserter, 340 of a plicator and spring system delivery device that will be discussed in greater detail below with regard to FIG. 5. Although first and second plicator systems 100A and 100B are shown to be the same size (e.g., length and diameter) in FIG. 3F, this need not always be the case. In some embodiments, a first and a second plicator system 100A and 100B may vary in size depending upon, for example, a tubular organ into which it is intended to be inserted and/or other factors such a patient anatomy and/or desired expansion rates of spring 320A or 320B.

A first end of spring 320A or 320B may be integrated into, or otherwise attached to, first plicator system 100A and a second end of spring 320A or 320B may be integrated into, or otherwise attached to, second plicator system 100B as shown in, for example, FIG. 3F. Spring 320A or 320B may be attached to first and/or second plicator system 100A and/or 100B via, for example, one or more interfacing with recess(es) within, for example, an outer diameter spring 320A or 320B that may be configured to cooperate with a corresponding extension projecting from inner component 130 of first and/or second plicator system 100A and/or 100B.

Plicator and spring system 300 and/or components thereof may come in a variety of sizes depending on, for example, patient anatomy, tubular organ type, and/or tubular organ diameter. Exemplary dimensions for an outer diameter of plicator/spring system 300 and/or components thereof may be within a range of, for example, 8 mm to 30 mm and exemplary lengths of plicator and spring system 300 while in a compressed state may be within a range of, for example, 8 mm-40 mm and when in an expanded state may be within 15 -100 mm. A spring force exerted by plicator/spring system 300 on a tubular organ in which it is implanted may be within a range of, for example 0.2 N-1.5 N and, in some cases, the spring force exerted on the tubular organ tissue may be proportional to a size of the plicator and spring system 300 being used. For example, when plicator and spring system 300 is 10 mm in diameter, when compressed, the maximum compressed spring force it may exert on tubular organ tissue may be in within a range of 0.25-0.45 N and may be within a range of 0.6-0.95 N for a plicator and spring system 300 that is the 20 mm in diameter.

Plicator and spring system 300 and/or components thereof may be made from any suitable material, including, but not limited to, a metal (e.g., stainless steel), a nickel-titanium alloy (nitinol), plastic, and/or a biodegradable/bioabsorbable or non-biodegradable polymer. Spring 320A and/or 320B may be made from any suitable material, including, but not limited to, a metal (e.g., stainless steel), a nickel-titanium alloy (nitinol), plastic, and/or a biodegradable/bioabsorbable or non-biodegradable polymer.

Exemplary biodegradable/bioabsorbable polymers include without limitation, polyarylates (L-tyrosine-derived or free acid), poly(a-hydroxy-esters), poly(-hydroxy-esters), polyamides, poly(amino acid), polyalkanotes, polyalkylene alkylates, polyalkylene oxylates, polyalkylene succinates, polyanhydrides, polyanhydride esters, polyaspartimic acid, polybutylene diglycolate, poly(caprolactone), poly(caprolactone)/poly(ethylene glycol) copolymers, poly(carbonate), L-tyrosine-derived polycarbonates, polycyanoacrylates, polydihidropyrans, poly(dioxanone), poly-p-dioxanone, poly(epsilon-caprolactone), poly(epsilon-caprolactone-dimethyltrimethylene carbonate), poly(esteramide), poly(esters), aliphatic polyesters, poly(etherester), poly(ethylene glycol)/poly(orthoester) copolymers, poly(glutarunic acid), poly(glycolic acid), poly(glycolide), poly(glycolide)/poly(ethylene glycol) copolymers, poly(glycolide-trimethylene carbonate), poly(hydroxyalkanoates), poly(hydroxybutyrate), poly(hydroxybutyrate-co-valerate), poly(imino carbonates), polyketals, poly(lactic acid), poly(lactic acid-co-glycolic acid), poly(lactic acid-co-glycolic acid)/poly(ethylene glycol) copolymers, poly(lactide), poly(lactide-co-caprolactone), poly(DL-lactide-co-glycolide), poly(lactide-co-glycolide)/poly(ethylene glycol) copolymers, poly(lactide)/poly(ethy1ene glycol) copolymers, poly(lactide)/poly(glycolide) copolymers, polyorthoesters, poly(oxyethylene)/poly(oxypropylene) copolymers, polypeptides, polyphosphazenes, polyphosphoesters, polyphosphoester urethanes, poly(propylene fumarate-co-ethylene glycol), poly(trimethylene carbonate), polytyrosine carbonate, polyurethane, PorLastin or silk-ealastin polymers, spider silk, tephaflex, terpolymer (copolymers of glycolide, lactide or dimethyltrimethylene carbonate), and combinations, mixtures or copolymers thereof. In one variation, the biodegradable polymer is polycaprolactone (PCL).

Examples of non-biodegradable polymers include, but are not limited to, poly(ethylene vinyl acetate), poly(vinyl acetate), silicone polymers, polyurethanes, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, and copolymers and blends thereof.

As shown in FIG. 3F, first and second plicator systems 100A and 100B are arranged in a first, open configuration with window 110 open and spring 320A and/or spring 320B is in a compressed, or closed, configuration as may be the case prior to implantation of plicator and spring system 300 within a tubular organ. Occluding component 310 may be configured as a component that occludes lumen 125 for first plicator system 100A so that, for example, negative air pressure (i.e., a vacuum) may be applied to plicator and spring system 300. Occluding component 310 may be, for example, an occlusion balloon, an elastomeric umbrella (e.g., with or without nitinol ribbing), a braided occlusion device, and/or a vascular occlusion device.

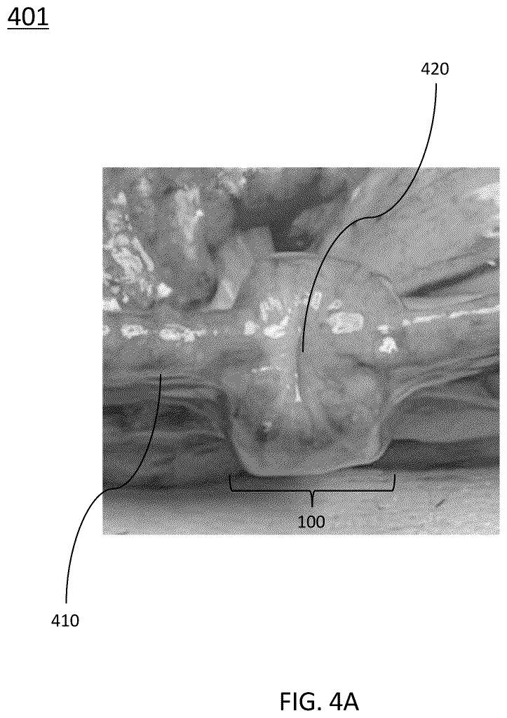

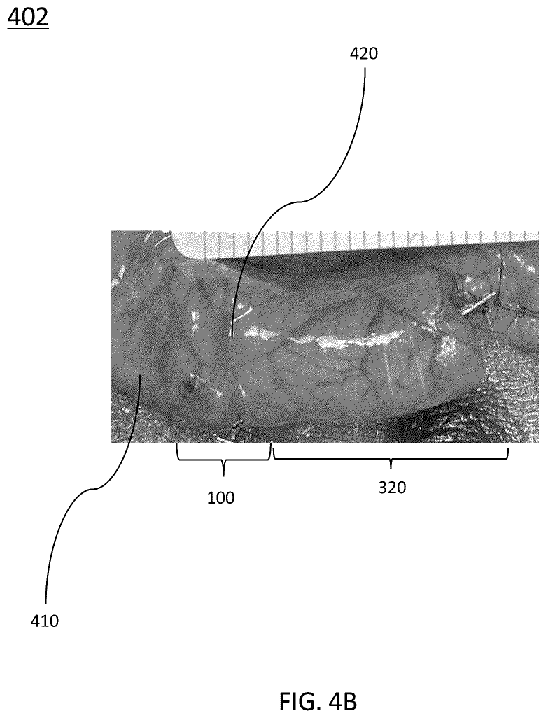

Following delivery of plicator and spring system 300 to a tubular organ (not shown), application of negative air pressure to plicator and spring system 300 may cause tissue of the tubular organ to be pulled into windows 110 of first and a second plicator system 100A and 100B wherein the tissue may engage with one or more teeth 115 of first and/or second plicator system 100A and/or 100B. On some occasions, a degree of negative air pressure may be applied to plicator and spring system 300 that is sufficient to account for anatomical variability in tissue when drawing the tissue into window 110. When in the second, closed configuration shown in FIG. 3, tissue may be grabbed by and retained within windows 110 by teeth 115 as shown in, for example, photographs 401 and 402 of FIGS. 4A and 4B, respectively, as described below.

Once tissue is positioned within windows 110, first and second plicator systems 100A and 100B may transition from the first, open, configuration shown in FIG. 3F to a second, closed, configuration as shown in FIG. 3G via for example, a pull-through wire, a breakaway fuse and/or a mechanical fuse feature. Additionally, or alternatively, first and second plicator system 100A and 100B may transition from the first, open, configuration shown in FIG. 3F to the second configuration as shown in FIG. 3G via application of negative air pressure to plicator and spring system 300 (i.e., first and/or second plicator system may be sucked closed via the application of a vacuum). Additionally, or alternatively, the transition of first and second plicator systems 100A and 100B from the first, open, configuration to the second, closed configuration may be triggered by actuation (e.g., squeezing together) of handles provided by a plicator and spring system 300 delivery device as shown in FIG. 5 and discussed below.

Once the tissue is secured, clamped, trapped, and/or pierced within closed window 110, the negative pressure may be decreased and/or stopped and occluding component 310 may be removed from plicator and spring system 300 as shown in FIG. 3H. Occluding component 310 may be removed from plicator and spring system 300 via, for example, pulling the occluding component partially, or completely, into a delivery device and/or first or second plicator systems 100A and 100B in a manner that does not, for example, impede disengagement of first plicator system 100A or second plicator system 100B from the delivery device. At this time, or concurrently with the removal of the occluding component 310, the delivery extension 340 may be withdrawn from within plicator system 100B

Over time, spring 320A and/or spring 320B may expand within the tubular organ in which it has been implanted thereby exerting a tensile and/or distending force on the tubular organ, which encourages and/or facilitates growth and/or elongation a portion of the tubular organ engaged with plicator and spring system 300. FIG. 3I is a schematic representation of the plicator and spring system 300 when coil is in an expanded, or open, configuration as may be the case 1-6 weeks following implantation of plicator and spring system 300 within the tubular organ.

FIG. 4A is a photograph 401 of a single plicator system 100 positioned within a tubular organ 410 that, on this occasion, is a small intestine. Photograph 401 shows a portion of invaginated tissue 420 of the tubular organ 410 that has been drawn into a window like window 110 and sealed therein via a transitioning of a plicator system 100 from an open configuration to a closed configuration.

FIG. 4B is a photograph of tubular organ tissue invaginated into a window of a plicator and spring system that includes one plicator system 100 and one spring 320A and/or spring 320B positioned within a tubular organ 410 that, on this occasion, is a small intestine. Photograph 402 shows a portion of invaginated tissue 420 of the tubular organ 410 that has been drawn into a window like window 110 and sealed therein via a transitioning of a plicator system 100 from an open configuration to a closed configuration and also shows spring 320A and/or spring 320B in an expanded state within the tubular organ.

FIG. 5 is a schematic representation of plicator and spring system 300 coupled to a plicator and spring system delivery device 500 that may include delivery extension 340, a vacuum/insufflation port 512, an occluding component control interface 514, an actuator 516, an accessory port 518, a first actuation handle 520A, and a second actuation handle 520B. Plicator and spring system delivery device 500 may be configured to enable a clinician (e.g., surgeon or doctor) to place a plicator and spring system 300 within a tubular organ via an opening therein. The opening in the tubular organ may be, for example, a surgical opening or a stoma (i.e., a portion of the tubular organ that extends from the body as is the case when, for example, a portion of the tubular organ extends out from the epidermis of a patient).

Vacuum/insufflation port 512 may be configured to couple to a vacuum or suction pump that may be configured to apply negative air pressure to plicator and spring system 300 and plicator and spring system delivery device 500 in order to, for example, pull tubular organ tissue into windows 110 as shown in, for example, photograph(s) 401 or 402 of FIGS. 4A and 4B, respectively. At this time, the actuator is applied which transitions first and/or second plication systems 100A and/or 100B from an open configuration, as shown in FIG. 3F, to a closed configuration as shown in FIG. 3G.

Accessory port 518 may be used for insertion of a secondary device (e.g., a camera, a fiberscope, and/or ablating device) by which to observe the delivery of plicator and spring system 300 to a target portion of a tubular organ and/or treat tissue.

A position of occluding component 310 may be controlled via occluding component control interface 514, which may be, for example, a handle for a wire or other device physically coupled to occluding component 310 that may be used (e.g., clinician can pull on a wire coupled to occluding component 310 via occluding component control interface 514 to extract occluding component 310 from plicator and spring system 300) to remove occluding component 310 from first plicator system 100A. In some embodiments, occluding component 310 may be pulled into delivery extension 340 following placement of plicator and spring system 300 within the tubular organ. In these embodiments, a scope or other device inserted into, for example, accessory port 518 may be used to, for example, visually and/or tactilely verify that occluding component 310 has been successfully removed from plicator and spring system 300 and/or is no longer in the tubular organ and/or surgical opening of the patient. In other embodiments, occluding component 310 may be pulled through component control interface 514 and out of plicator and spring system delivery device 500.

Delivery extension 340 may be of a length sufficient to insert the plicator and spring system 300 into the tubular organ. On some occasions, delivery extension 340 may be relatively short (e.g., 7 cm-17 cm) as may be needed when inserting plicator and spring system 300 into an infant or long (e.g., 17 cm-25 cm), as may be needed when inserting plicator and spring system 300 into an adult. Delivery extension 340 may be rigid and/or flexible and may be hollow so that, for example, negative pressure applied to vacuum/insufflation port 512 may be communicated to plicator and spring system 300 and/or occluding component 310 may be sucked, or otherwise pulled, into delivery extension 340. Delivery extension 340 may also be configured so that a wire or other device coupled to occluding component 310 and/or occluding component control interface 514 may be positioned therein and may move through delivery extension 340. Additionally, or alternatively, delivery extension 340 may be configured so that a mechanism (pull-through wire, a breakaway fuse and/or a mechanical fuse) configured to transition first and second plicator systems 100A and 100B from the first, open, configuration to the second, closed configuration may be positioned within delivery extension 340 and/or may move through delivery extension 310. Additionally, or alternatively, delivery extension 340 may be configured so that a scope or other device passing through accessory port 518 may be positioned therein and may move through delivery extension 340.

An end of delivery extension 340 may be configured to couple to second plicator system 100B via an extension that fits into a corresponding notch in inner component 130 as shown in FIGS. 3F and 3G. Second plicator system 100B and/or plicator and spring system 300 may be released from delivery extension 340 via manual manipulation (e.g., squeezing) of actuator 516. In some embodiments, actuation (e.g., squeezing together) of first and second actuation handles 520A and 520B may cause the transition of first and second plicator systems 100A and 100B from the first, open, configuration to the second, closed configuration.

Once placed within a tubular organ (e.g., first and second plication systems 100A and 110B) have invaginated tubular organ tissue into their respective windows and are in a closed configuration (see e.g., FIG. 3G), thereby retaining the invaginated tissue within the closed windows), plicator and spring system 300 may begin to transition from a compressed state as shown in FIG. 3H to an expanded state as shown in FIG. 3I within the tubular organ thereby facilitating growth of the tubular organ tissue that may result in an elongation of the tubular organ. Plicator and spring system 300 may be removed and/or evacuated from the tubular organ via, for example, bio-absorption of one or more components of plicator and spring system 300, natural sloughing off of tissue within the tubular organ that releases the tissue held within closed windows 110 and/or by teeth 115 so that plicator and spring system 300 may pass through the digestive system of the patient and be evacuated from the patient's body, and/or surgical removal of plicator and spring system 300 or a portion thereof.

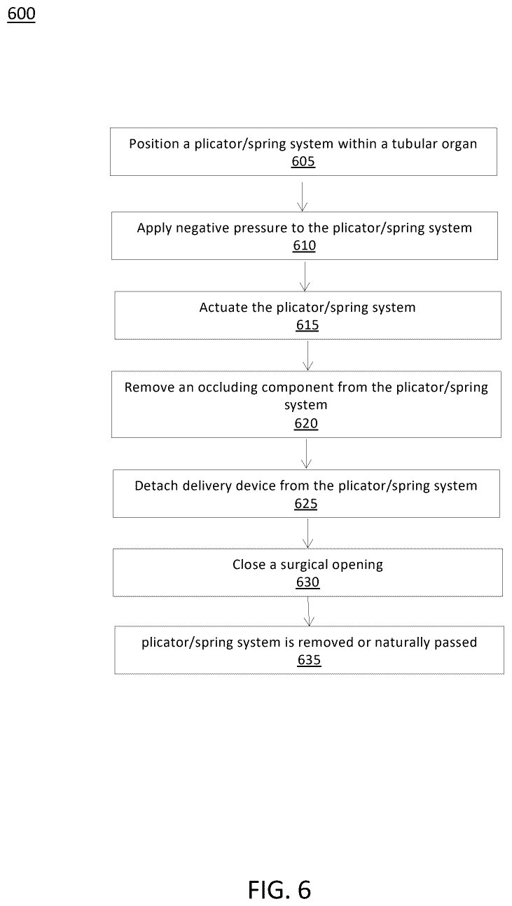

FIG. 6 is a flowchart illustrating an exemplary process 600 for treating a patient using a plicator and spring system as disclosed herein. Process 600 may be executed by, for example, a medical treatment provider, such as a surgeon or doctor, and/or a group of medical treatment providers.

Initially, in step 605, a plicator and spring system such as plicator and spring system 300 may be positioned within a tubular organ. Step 605 may be executed via, for example, use of a plicator and spring system delivery device such as plicator and spring system delivery device 500 to insert the plicator and spring system into a surgical opening in the patient's tubular organ. Alternatively, step 605 may be executed via a minimally invasive and/or endoscopic procedure. Alternatively, step 605 may be executed trans-anally and or trans-orally via, for example, use of an endoscopic-like device to position the plicator and spring system in a target location within the subject tubular organ.

Next, in step 610, negative pressure (i.e., a vacuum) may be applied to the plicator and spring system to secure the plicator and spring system within the tubular organ via, for example, clamping and/or piercing of the tissue. In some cases, execution of step 610 may operate to pull tubular organ tissue into a window, such as window 110, of a plicator system such as plicator system 100, thereby invaginating the tubular organ tissue within the window.

In step 615, the plicator and spring system may be actuated so that the plicator systems included therein transition from a first, open, configuration (see e.g., FIG. 3F), to a second, closed, configuration (see e.g., FIG. 3G) thereby sealing, or retaining, tissue invaginated into open windows of the plicator systems within the closed windows. In some embodiments, step 615 may be executed via use of actuation handles of a delivery device such as actuation handles 520A and 520B of plicator and spring system delivery device 500 as described above with regard to FIG. 5. Additionally, or alternatively, the negative pressure applied in step 610 may translate the plicator and spring systems from an open arrangement (see e.g., FIG. 3F) to a closed arrangement (see e.g., FIG. 3G), thereby securing tubular organ tissue sucked into the windows within the closed windows.

When plicator and spring system includes an occluding component such as occluding component 310, or some other device, to facilitate creation of a vacuum within the plicator and spring system, the occluding component may be removed from the plicator and spring system following deployment within the tubular organ, thereby opening a lumen of the plicator and spring system so that material (e.g., bodily fluids, digested food, water, etc.) may flow through the plicator and spring system positioned within the tubular organ (step 620).

In step 625, a delivery device (if used) may then be detached from plicator and spring system and the delivery device may be removed from the tubular organ and patient. Optionally (e.g., in situations where process 600 is being executed during an open surgical procedure), the surgical opening may then be closed (step 630) and process 600 may end. In some embodiments, steps 605-625 may be repeated to position multiple plicator and spring systems within a tubular organ concurrently or serially (when done serially, repeated execution of step 630 may also be necessary).

In some embodiments, the plicator and spring system may naturally disengage from the tubular organ over time via, for example, a sloughing off of tubular organ tissue invaginated into the windows of the plicatory and spring system and, once disengaged, may pass through the patient's digestive tract. When this does not occur, or when otherwise necessary, the plicator and spring system may be removed from the patient via, for example, surgery (step 635). In many cases, the natural passing of the plicator and spring system or surgical removal of the plicator and spring system may occur 1-6 weeks execution of step 605 when, for example, a target amount enterogenesis of the tubular organ has occurred.

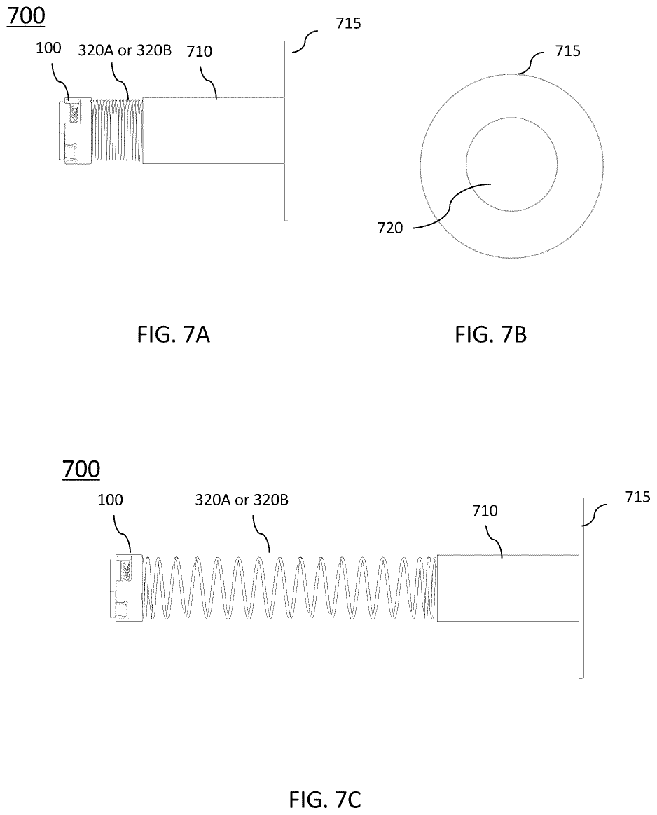

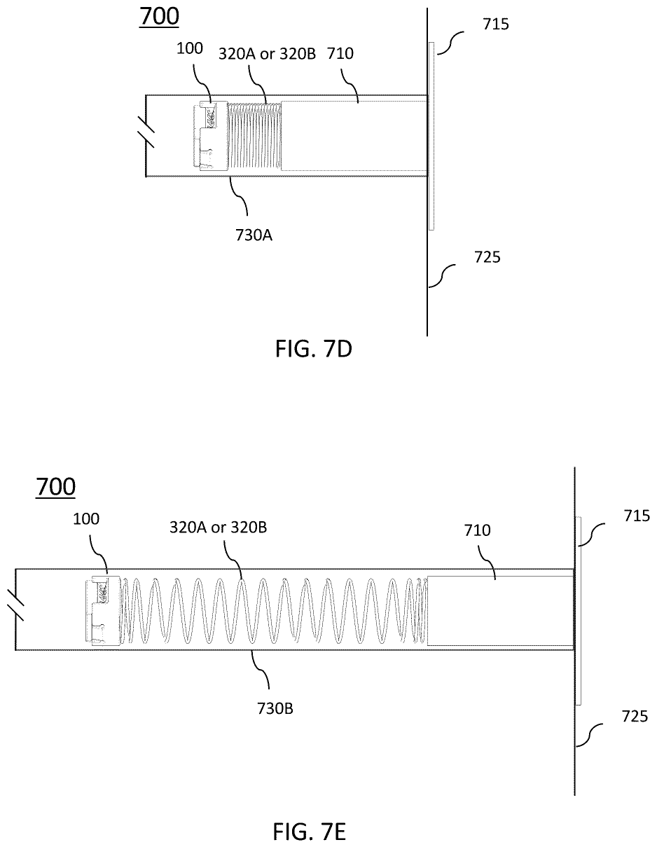

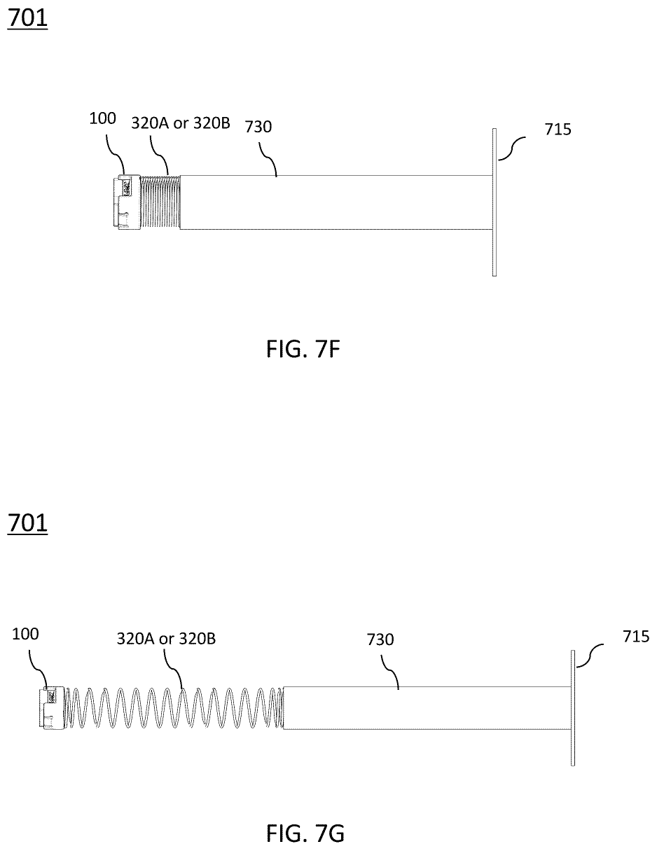

FIG. 7A-7G are schematic diagrams of plicator, spring, and flange system systems for delivery to a tubular organ through an opening and/or stoma (e.g., a functional or defunctional limb of the small intestine extending through the abdomen) of a patient. FIG. 7A provides a side view of a plicator, spring, and flange system system 700 that includes a plicator system 100, a spring 320, and a tube 710 with a flange 715. In some embodiments, plicator system 100, spring 320, and tube 710 with flange 715 may be a one-piece system (e.g., fused together) and, in other embodiments, the plicator, spring, and flange system system 700 may include separate plicator system 100, spring 320, and tube 710 with a flange 715 components that are assembled prior to an implantation procedure. FIG. 7D provides a side view of plicator, spring, and flange system system 700 when implanted in a subject with an epidermis 725 and a tubular organ 730A in an unextended state.

FIG. 7B provides a front view of flange 715 showing an opening 720 that aligns with a lumen of tube 710 and a tubular organ into which plicator, spring, and flange system system 700 is inserted via the stoma or opening in, for example, the abdominal wall until flange 715 abuts epidermis 725. Tube 710 and/or flange 715 may be configured to provide an anchoring surface against which spring 320A and/or spring 320B may exert an expansive force to elongate tubular organ 730A to an elongated tubular organ 730B as shown in FIG. 7E (which is a side view of plicator, spring, and flange system system 700 when implanted in the subject with an elongated tubular organ 730B) and described herein. In some embodiments, an adessive may be applied to the epidermis-facing side of flange 715 and/or the epidermis proximate to the opening into which plicator, spring, and flange system system 700 is inserted to hold plicator, spring, and flange system system 700 in place. Additionally, or alternatively, an adhesive mechanism (e.g., tape or a strap) may be applied to the outside of flange 715 to hold plicator and spring system 700 in place. Following insertion, plicator system 100 may engage with/affix to tissue of the tubular organ in a manner described herein with, reference to, for example, FIGS. 3F-5. Once seated within the tubular organ, spring 320A and/or spring 320B may expand, as shown in FIGS. 7C and 7E to elongate the tubular organ as, for example, shown in FIG. 7E and described herein.

Plicator, spring, and flange system system 700 may be inserted into a stoma or opening in, for example, the abdominal wall until flange 715 meets the epidermis of the patient. In some embodiments, tube 710 may be manufactured to be flexible and relatively soft so that it does not impede motion of the patient or cause him or her discomfort. In some embodiments, an inserter (e.g., delivery extension 340) that is flexible, yet rigid, may be removably inserted through a central lumen of plicator, spring, and flange system system 700 prior to placement within the tubular organ. The inserter may operate to provide sufficient rigidity to plicator, spring, and flange system system 700 so that it maintains structural integrity when inserted (e.g., doesn't collapse or fold) into the tubular organ. In some embodiments, the inserter may also operate to remove occluding component 310 and/or actuate plicator system 100 from an open to a closed state as described herein.

In embodiments where plicator, spring, and flange system system 700 comprises separate components, plicator system 100 may first be inserted into the tubular organ and affixed thereto as described herein. Then, spring 320A and/or spring 320B (in a compressed and/or expanded state) may be inserted into the tubular organ until it abuts plicator system 100. When in an expanded state, spring 320A and/or spring 320B may be compressed via force exerted thereon by tube 710 with flange 715 when it is inserted into the tubular organ until flange 715 abuts the skin of the patient.

FIGS. 7F and 7G provide side views of another exemplary plicator, spring, and flange system system 701 configured to be inserted through an opening in the skin or body in a manner similar to plicator, spring, and flange system 700. Plicator and spring system 701 includes plicator system 100, spring 320, and an elongate tube 730 with flange 715. Plicator and spring system 701 may be deployed in situations where placement of plication system 100 and/or expansion of the tubular organ further into the body (as compared with plicator and spring system 700) is desired. Additionally, or alternatively plicator and spring system 701 may be used serially after plicator and spring system 700 is used, and the tubular organ has been extended to further extend the tubular organ as shown in, for example, FIG. 7E. In these embodiments, elongate tube 730 may be inserted into the tubular organ and may compress (or recompress) spring 320A and/or spring 320B so that a plicator system 100 and spring 320A and/or spring 320B already positioned within a tubular organ may be reused to further extend, or elongate, the tubular organ in, for example, a serial (e.g., 2-6 times) fashion. Alternatively, spring 320A and/or spring 320B may be removed from the tubular organ and replaced with a different spring (e.g., a spring similar to spring 320A and/or spring 320B and/or a spring with one or more characteristics (e.g., size, spring force, etc.) different from spring 320). Then, elongate tube 730 with flange 715 may be inserted into the tubular organ to abut (when the second spring is already compressed) and/or compress (when the second spring is uncompressed) and then abut the second spring when plicator, spring, and flange system system is fully seated within the patient.—may be placed in a subject in a manner similar to that described above with regard to, for example, FIGS. 7D and 7E.

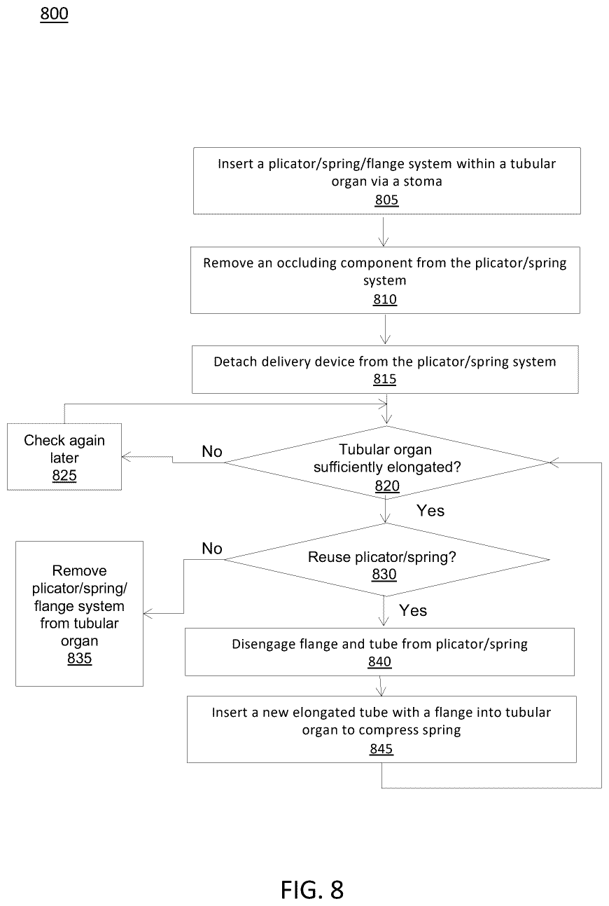

FIG. 8 is a flowchart illustrating a process 800 for treating a patient using a plicator, spring, and flange system system such as plicator, spring, and flange system system 700 and/or plicator, spring, and flange system system 701 that may be inserted into a tubular organ via an exposed opening in a tubular organ (e.g., small intestine or large intestine) and/or a stoma. Process 800 may be executed by, for example, a medical treatment provider and/or a group of medical treatment providers.

Initially, in step 805, a plicator, spring, and flange system system such as plicator, spring, and flange system system 700 or 701 may be positioned within a tubular organ via insertion through an opening in a patient such as a stoma. Step 805 may be executed via, for example, use of a plicator, spring, and flange system system delivery device such as plicator and spring system delivery device 500 to insert the plicator, spring, and flange system system into a surgical opening in the patient's tubular organ. Alternatively, step 805 may be executed using an inserter device positioned within a lumen of the plicator, spring, and flange system system that provides structural rigidity to the plicator, spring, and flange system system and/or allows for easier manipulation of plicator, spring, and flange system system within the tubular organ.

In some embodiments, when, for example, plicator, spring, and flange system system includes a plurality of components, execution of step 805 may include multiple sub-steps including, but not limited to, insertion of a plicator such as plicator system 100 into a tubular organ through the stoma and affixing the plicator to the tubular organ as described herein (e.g., step 610 and 615). Next, the spring, like spring 320, of the plicator, spring, and flange system system may be inserted into the tubular organ so that the spring abuts the plicator. In some instances, the spring may be compressed prior to insertion into the tubular organ. In other embodiments, the spring may be uncompressed prior to insertion into the tubular organ. In these embodiments, the spring may be compressed via insertion of a stoma interface such as tube 710 and flange 715 into the tubular organ until it compresses the spring to a desired length.

When the plicator, spring, and flange system system includes an occluding component such as occluding component 310, or some other device, to facilitate creation of a vacuum within the plicator, spring, and flange system system, the occluding component may be removed from the plicator, spring, and flange system system thereby opening a lumen of the plicator, spring, and flange system system so that material (e.g., bodily fluids, digested food, water, etc.) may flow through the plicator, spring, and flange system system positioned within the tubular organ (step 810).

In step 815, a delivery device (if used) may then be detached from the plicator, spring, and flange system system and the delivery device may be removed from the tubular organ and patient. In step 820, it may be determined whether the tubular organ has been sufficiently elongated by a distending force exerted by the plicator, spring, and flange system system on the tubular organ. Step 820 may be executed, for example, 1-5 weeks following insertion of the plicator, spring, and flange system system. When the tubular organ has not been sufficiently elongated, an attending physician or medical staff may check again later (step 825) until the tubular organ is sufficiently elongated.

When the tubular organ is sufficiently elongated, it may be determined whether, or not, to reuse the plicator and/or spring already in position within the tubular organ to further elongate the tubular organ and, if not, the plicator, spring, and flange system system may be removed from the tubular organ and/or patient (step 835). If so, then a flange of the plicator, spring, and flange system system may be disengaged from the spring and, in some cases, the spring may be disengaged from the plicator (step 840) and a new spring and/or elongated tube with a flange may be inserted into the tubular organ so that the spring abuts the plicator and is compressed so that the spring and the plicator, spring, and flange system system may further exert distending force on the tubular organ Steps 820-845 may be repeated multiple times to further elongate the tubular organ so long as the plicator is still in position and functioning as intended.

The present disclosure further provides a kit that includes, for example, one or more plicator and spring system(s) 300 that, in some cases may be of various sizes as well as a plicator and spring system delivery device such as plicator and spring system delivery device 500. At times, when a patient may be treated with a plurality of plicator and spring systems 300, for example, the kit may include a number of plicator and spring systems 300 for serial and/or concurrent use to expand the patient's tubular organ to a degree greater than is possible with just a single plicator and spring system 300. When a plurality of plicator and spring systems 300 are concurrently placed within a patient's tubular organ, they may be placed, for example, every 2-4 inches along a length of the patient's tubular organ.

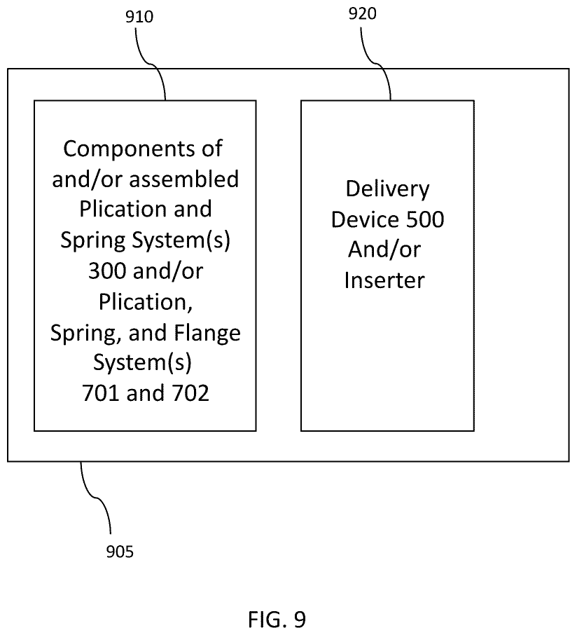

FIG. 9 provides a diagram of one exemplary kit 900 that includes a housing 905, a first container 910, and a second container 920. It will be understood that the components of kit 900 may be Housing 905 may be any appropriate housing configured to hold first and second containers 920 such as a plastic tray and/or a bag. In many instances, housing 905 may be sterile and/or may be configured to keep contents of first and second containers 920 sterile and dry. First and/or second containers may be, for example, a bag and/or plastic tray.

First container 910 may include, or contain, one or more plication and spring systems 300, components of plication and spring systems 300, one or more plication, spring, and flange system 701 and/or 701, and/or components of one or more plication, spring, and flange system 701 and/or 701. In some cases, first container 910 may contain one or more plication and spring systems 300, components of plication and spring systems 300, one or more plication, spring, and flange system 701 and/or 701, and/or components of one or more plication, spring, and flange system 701 and/or 701 may be of different sizes and/or configured for use in different situations including, but not limited to, implantation in an adult, implantation in a child, and/or components that may be assembled on demand according to, for example, clinical need and/or physician preference.

Second container 920 may include a delivery device such as delivery device 500 and/or an inserter configured for use with, for example, plicator, spring, and flange system 701 and/or 701 and/or components needed for serial implantation of one or more plicator and spring systems as disclosed herein.

1. A plicator system configured for implantation within a tubular organ, the plicator comprising:
an outer component including a first lumen, a first notch, and a retention mechanism; and
an inner component including a second lumen, a second notch, and a plurality of projections extending from an outer surface of the inner component, each of the plurality of extensions being arranged and configured to engage with the retention mechanism,
wherein a portion of the inner component is disposed within the outer component so that the first lumen aligns with the second lumen, thereby generating a central lumen for the plicator system,
wherein the first notch and the second notch are arranged and configured to form a window with an open area into which a portion of tubular organ tissue may be invaginated when the plicator system is implanted into the tubular organ and the plicator system is arranged in an open configuration,
wherein the plicator system is configured to translate from the open configuration to a closed configuration as the inner component is pushed toward outer component, thereby reducing a size of the open area of the window and clamping the invaginated tubular organ tissue therein so that the plicator system is securely implanted within the tubular organ.

2. The plicator system of claim 1, wherein the outer component includes one or more teeth that extend into the first notch, the one or more teeth being configured to pierce tubular organ tissue invaginated into the window.

3. The plicator system of claim 1, wherein the inner component includes one or more teeth that extend into the second notch, the one or more teeth being configured to pierce tubular organ tissue invaginated into the window.

4. The plicator system of claim 3, wherein a size of the one or more teeth of at least one of the inner component and the outer component is responsive to at least one of a size and a thickness of the tubular organ.

5. The plicator system of claim 1, wherein the retention mechanism is a clip mechanism configured to engage with each projection of the plurality of projections as the plicator system translates from the open configuration to the closed configuration.

6. The plicator system of claim 1, wherein the retention mechanism is a clip mechanism configured to engage with each projection of the plurality of projections as the plicator system translates from the open configuration to the closed configuration, thereby locking the inner component in place relative to outer component.

7. The plicator system of claim 1, wherein the retention mechanism is a ratcheting mechanism.

8. The plicator system of claim 1, wherein the retention mechanism comprises a cantilevered snap configured to engage with a projection of the plurality of projections and maintain a position of inner component relative to outer component.

9. The plicator system of claim 1, further comprising:
an occluding component, the occluding component being attached to an end of the inner component not disposed within the outer component and configured to occlude the second lumen.

10. The plicator system of claim 1, further comprising:
a delivery extension, the delivery extension being attached to an end of the inner component not disposed within the outer component, the delivery extension being configured for cooperation with a plicator system delivery device.

11. The plicator system of claim 1, further comprising:
a spring, the spring being attached to an end of the outer component in which the inner component is not disposed.

12. The plicator system of claim 1, wherein the inner component and the outer component are cylindrical.

13. A plicator and spring system comprising:
a first plicator system comprising:
a first outer component including a first lumen, a first notch, and a retention mechanism; and
a first inner component including a second lumen, a second notch, and a plurality of projections extending from an outer surface of the inner component, each of the plurality of extensions being arranged and configured to engage with the retention mechanism,
wherein a portion of the inner component is disposed within the outer component so that the first lumen aligns with the second lumen, thereby generating a central lumen for the plicator system,
wherein the first notch and the second notch are arranged and configured to form a first window with an open area into which a portion of tubular organ tissue may be invaginated when the plicator system is implanted into the tubular organ and the plicator system is arranged in an open configuration,
wherein the plicator system is configured to translate from the open configuration to a closed configuration as the first inner component is pushed toward outer component, thereby reducing a size of the open area of the first window and clamping the invaginated tubular organ tissue therein so that the plicator system is securely implanted within the tubular organ;
a second plicator system comprising:
a second outer component including a third lumen, a third notch, and a retention mechanism; and
a second inner component including a fourth lumen, a fourth notch, and a plurality of projections extending from an outer surface of the second inner component, each of the plurality of extensions being arranged and configured to engage with the retention mechanism,
wherein a portion of the second inner component is disposed within the second outer component so that the third lumen aligns with the fourth lumen, thereby generating a central lumen for the second plicator system,
wherein the first notch and the second notch are arranged and configured to form a second window with an open area into which a portion of tubular organ tissue may be invaginated when the plicator system is implanted into the tubular organ and the plicator system is arranged in an open configuration,
wherein the plicator system is configured to translate from the open configuration to a closed configuration as the second inner component is pushed toward outer component, thereby reducing a size of the open area of the second window and clamping the invaginated tubular organ tissue therein so that the plicator system is securely implanted within the tubular organ; and
a spring having a tubular body with a lumen therethrough that aligns with the first, second, third, and fourth lumen, the spring being attached to the first outer component and the second outer component and disposed therebetween.

14. The plicator and spring system of claim 13, wherein the spring is configured to axially expand, thereby pushing the first plicator system away from the second plicator system.

15. The plicator and spring system of claim 13, wherein the spring is an axially expanding spring.

16. The plicator and spring system of claim 13, wherein a size of the first plicator system is different from a size of the second plicator system.

17. The plicator and spring system of claim 13, further comprising:
an occluding component, the occluding component being attached to an end of the first inner component not disposed within the first outer component and configured to occlude the second lumen.

18. The plicator and spring system of claim 13, further comprising:
a delivery extension, the delivery extension being removably attached to an end of the second inner component not disposed within the second outer component and configured to attach to a delivery device for the plicator and spring system.

19. The plicator and spring system of claim 18, further comprising:
a delivery device coupled to the delivery extension, the delivery device being configured to facilitate invagination of tubular organ tissue into the first window of the first plicator system and the second window of the second plicator system and facilitate closing of the first window and the second window, thereby securing the tubular organ tissue invaginated into the first window and the second window therein.

20. A method for implanting a plicator and spring system into a tubular organ of a subject, the method comprising:
positioning the plicator and spring system within the tubular organ;
applying negative pressure to the plicator and spring system, thereby pulling tubular organ tissue into a window of the plicator and spring system; and
actuating the plicator and spring system to secure the tubular organ tissue within the window.

21. The method of claim 20, wherein the plicator and spring system includes a lumen and an occluding component configured to occlude the lumen, the method further comprising:
removing the occluding component following implantation of the plicator and spring system within the tubular organ.

22. (canceled)

23. The method of claim 20, wherein the plicator and spring system includes a delivery extension configured to cooperate with the plicator and spring system delivery device, the method further comprising:
removing the delivery extension following actuation of the plicator and spring system.
